(19) United States
(12) Patent Application Publication
(10) Pub. No.: US 2011/0015512 A1
(43) Pub. Date: Jan. 20, 2011

(54) MEASURING OUTFLOW RESISTANCE/FACILITY OF AN EYE

(75) Inventors: Tingrui Pan, Woodland, CA (US); James David Brandt, Carmichael, CA (US)

Correspondence Address: FENWICK & WEST LLP, SILICON VALLEY CENTER, 801 CALIFORNIA STREET, MOUNTAIN VIEW, CA, 94041, US

(73) Assignee: The Regents of the University of California, Oakland, CA (US)

(21) Appl. No.: 12/920,587

(22) PCT Filed: Mar. 6, 2009
(86) PCT No.: PCT/US09/36378

§ 371 Date: Sep. 1, 2010

Related U.S. Application Data
(60) Provisional application No. 61/034,484, filed on Mar. 6, 2008.

(51) Int. Cl. A61B 3/16

(52) U.S. Cl. 600/399

(57) ABSTRACT

A measurement system takes measurements of intraocular pressure and displaced ocular volume for determination of aqueous outflow resistance A device with a πgid outer wall, a flexible inner wall, and an inflatable bladder in between is placed over the eye A pressure measurement system is coupled to the bladder and is configured to measure a pressure of fluid within the bladder A hydraulic unit is coupled to the bladder and configured to control a flow of fluid between the bladder and an external reservoir, and to measure a change of volume in the bladder created by the pressure applied to the eye Both the pressure measurement system and hydraulic unit are directly controlled by and communicated with a microprocessor/computer In addition, the microprocessor computes the outflow resistance of the eye as a function of the pressure in the bladder and the change of volume in the bladder over time.

### CROSS REFERENCE TO RELATED APPLICATIONS

This application claims the benefit of U.S. Provisional Application No. 61/034,484, filed Mar. 6, 2008, the entire disclosure of which is hereby incorporated by reference in its entirety, including any appendices, for all purposes.

### BACKGROUND OF THE INVENTION

1. Field of the Invention

The invention relates generally to intraocular pressure measurement, and more specifically to medical systems and methods for measuring aqueous outflow resistance/facility of an eye.

2. Description of the Related Art

For more than a century, tonometry has been used to evaluate intraocular pressure (IOP), or fluid pressure inside an eye, which is considered to be the most important clinical risk factor for glaucomatous eyes. Eyes produce a watery fluid, or aqueous humor, that normally enters the eye and then drains out via an aqueous drainage pathway (e.g., the trabecular meshwork, uveoscleral pathways and episcleral veins) into the bloodstream. Glaucoma, an eye disease that can damage eyes and potentially result in blindness, causes a buildup of fluid inside the eye that does not drain properly due to problems in the drainage path and puts damaging pressure on the optic nerve.

Tonometry, the measurement of tension or pressure, can be used to evaluate this intraocular pressure and detect glaucoma by application of an instrument called a tonometer. One type of tonometry, indentation tonometry, measures the depth of an indentation produced in the cornea by a small plunger-like instrument. The amount of weight needed for indentation determines the IOP of the eye. Tonography, developed based on indentation tonometry, is a continuous tracking technology for monitoring the indentation level of an eye. Tonography is used to record changes in IOP due to sustained pressure on the eyeball. Tonography has been used to assess outflow resistance (or outflow facility) in the aqueous drainage path. Relating the indentation level to both intraocular pressure (Pₒ) and displaced ocular volume (ΔV), the aqueous outflow resistance (R) can be estimated by: R=ΔP/ΔV/Δt. Accurately measuring outflow resistance could potentially lead to a better understanding of the glaucomatous pathology. However, due to the invasiveness and length of the tonography procedure, as well as its highly imprecise nature, the procedure has not been used extensively in clinical practices since its original introduction in 1950s.

Current tonography procedures also encounter an intrinsic technical hurdle. In order to measure flow resistance or facility, two measurable quantities are typically required: pressure drop (ΔP) and flow rate (Q) or rate of volume change (ΔV/Δt). But in tonography, the only measurement made is through the reading of indentation level. Therefore, statistical correlations applied in tonography procedures relate the indentation levels to both volume change and pressure reading under a constant weight on the cornea surface. Jonas Friedenwald's early work in 1947 in this field provided the foundation of the methods. Although flow resistance can be “calculated” in this manner (under serially unreliable assumptions and limitedly studied correlations), the conclusion is neither mathematically nor physically convincing. In addition to the unreliability of the underlying principle itself, current tonography is also significantly affected by limited reproducibility. This instability of the measurement can result from that inconstant perturbing force (weight load) on the cornea surface, rapid eye movement-induced IOP variation, eyelid movement and squeezing-induced disturbances, etc.

With the recent developments in measurement sciences and polymer materials, the emerging flexible electronics and touch sensing techniques demonstrate great potential in biological and clinical applications. Accordingly, embodiments of the invention provide a safe, convenient, noninvasive and accurate measurement solution for a better assessment of aqueous outflow resistance, compared to the original concept of tonography.

### SUMMARY OF THE INVENTION

Embodiments of the invention provide methods, systems, and computer products for measuring the outflow resistance/facility of an eye. One embodiment of the system includes a contact-lens device comprising a rigid outer wall, a flexible inner wall, and an inflatable bladder disposed there between. The contact-lens device has a concave shape to allow placement over the eye, and the flexible inner wall contacts the eye. The system also includes a hydraulic unit coupled to the bladder and configured to control a flow of fluid between the bladder and an external reservoir. The hydraulic unit is further configured to measure a change of volume in the bladder over time. The system also includes a pressure measurement system coupled to the bladder and configured to measure a pressure of fluid within the bladder. In addition, the system includes computer-controlled logic configured to compute the outflow resistance of the eye as a function of the pressure in the bladder and the change of volume in the bladder over time

One embodiment of the method for measuring an outflow resistance of an eye comprises applying pressure to the eye and measuring the applied pressure to the eye. The method further includes directly measuring a volume change of the eye at a plurality of times and computing an outflow rate of fluid from the eye based on the measured volume change of the eye over time. In addition, the method includes determining the outflow resistance of the eye as a function of a ratio of the applied pressure and the outflow rate.

An embodiment of the computer program product for measuring an outflow resistance of an eye comprises a computer-readable storage medium containing computer program code. The code includes instructions for receiving a pressure measurement representing an applied pressure to the eye, and receiving a set of volume measurements representing a directly measured volume change of the eye at a plurality of times. The instructions further include computing an outflow rate of fluid from the eye based on the measured volume change of the eye over time. In addition, the instructions comprise determining the outflow resistance of the eye as a function of the ratio of the applied pressure and the outflow rate, and further using a biomechanical model of the eye to model dynamic effects.

The features and advantages described in this disclosure and in the following detailed description are not all-inclusive, and particularly, many additional features and advantages will be apparent to one of ordinary skill in the relevant art in view of the drawings, specification, and claims hereof. Moreover, it should be noted that the language used in the specification has been principally selected for readability and instructional purposes, and may not have been selected to delineate or circumscribe the inventive subject matter, resort to the claims being necessary to determine such inventive subject matter.

### BRIEF DESCRIPTION OF THE DRAWINGS

These and other features, aspects, and advantages of the present invention will become better understood with regard to the following description, and accompanying drawings, where:

FIG. 1a is an illustration of the dual measurement system for applying tonographic techniques to an eye, according to an embodiment.

FIG. 1b is an illustration of the dual measurement system for applying tonographic techniques to an eye, according to an embodiment.

FIG. 2 is a flowchart illustrating the steps performed by the dual measurement system, according to an embodiment.

FIGS. 3a and 3b are flowcharts illustrating other embodiments of the steps performed by the dual measurement system.

FIG. 4a is a depiction of the current principles of tonography.

FIG. 4b is an illustration of the dynamic, dual-parameter measurement (a) prior to and (b) after the ocular volume change, according to an embodiment.

FIG. 4c is an illustration of a lumped element circuit representation of the microfluidic model in the anterior chamber of the eye, according to an embodiment.

FIG. 5 is a high-level block diagram illustrating a standard computer system 200 for use with the invention.

FIG. 6 is a high-level block diagram illustrating the functional modules within the biomechanical modeling module 600, according to an embodiment.

FIG. 7 is a flowchart illustrating steps performed by the biomechanical modeling module 600, according to an embodiment.

FIG. 8a is an illustration of the microfabrication of the contact lens device, according to an embodiment.

FIGS. 8b and 8c are photographs of a prototype of an array of miniature pressure sensors fabricated onto a flexible contact-lens platform, according to an embodiment

FIGS. 9a and 9b are photographs of in vitro biomechanical test setups to evaluate the aqueous outflow resistance, according to an embodiment.

FIG. 9c is photograph of a prototype of the dynamic dual measurement system using the volume-adjustable contact-lens device, according to an embodiment.

### DETAILED DESCRIPTION OF THE INVENTION

### System for Outflow Resistance Measurement

The dual measurement system and method described here are generally based on the tonography principles, but with real-time, continuous and direct measurement on both intraocular pressure (Pₒ) and displaced ocular volume (ΔV). In comparison with the convention tonography technique that uses statistical correlations to calculate IOP and volume change from an indentation indicator, the dynamic, dual-parameter (ΔIOP-ΔV) measurement system detects both IOP and ocular volume changes simultaneously, and measures the outflow resistance directly in a short duration (e.g., a few minutes). An explanation of the principles behind tonography is provided in the appendix of U.S. Provisional Application No. 61/034,484, filed Mar. 6, 2008, which is incorporated by reference.

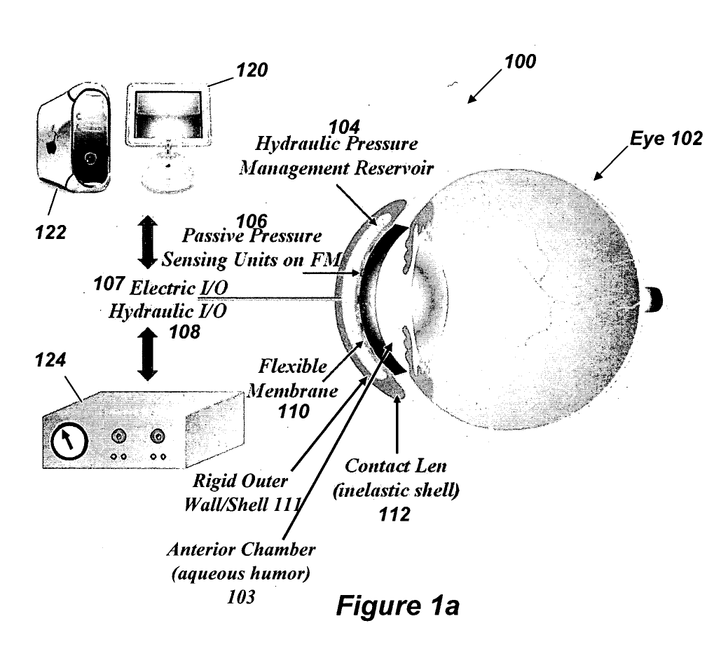

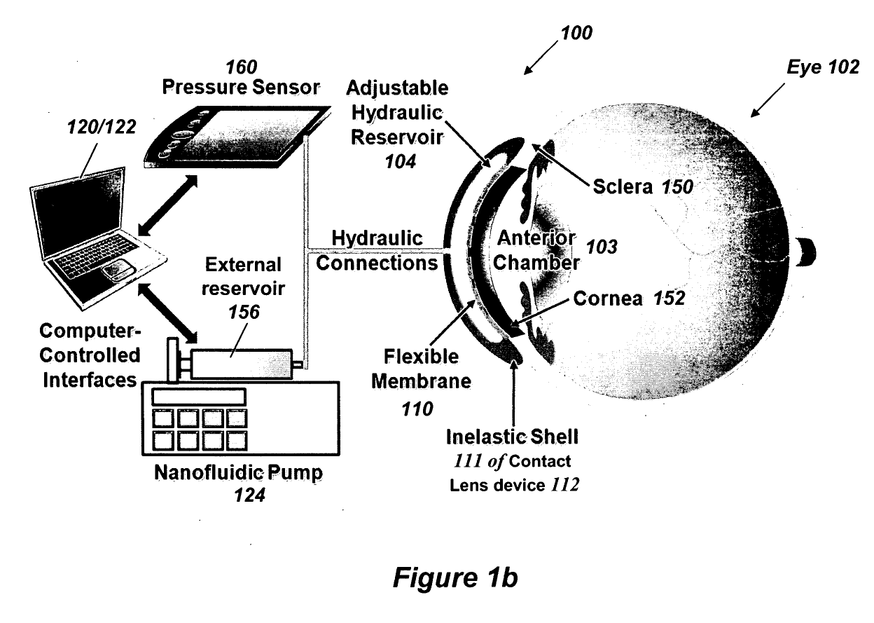

FIGS. 1a and b illustrate an eye 102 and further show the anterior chamber 103 of the eye 102 containing the aqueous humor, the sclera 150, and the cornea 152 of the eye 102. As explained above, eyes produce this watery fluid, or aqueous humor, that normally enters the eye and then drains out via an aqueous drainage pathway. However, in eyes with glaucoma, the aqueous humor typically does not drain properly creating pressure in the eye 102 that leads to vision problems. Thus, it is the properties associated with the flow of this aqueous humor that the dual measurement system and method described here will measure.

Along with the illustration of the eye 102, FIG. 1a further illustrates an embodiment of the contact lens-based dual measurement system 100 comprising an inflatable bladder, referred to in FIG. 1 as a hydraulic pressure management reservoir 104. The reservoir 104 is disposed between a rigid outer wall 111 (inelastic shell) and a flexible inner wall, referred to in FIG. 1 as a flexible membrane 110, forming a contact lens device 112 with a concave shape that can be placed in an eye 102 in a manner similar to a standard contact lens. When placed over the eye 102, the flexible inner wall/membrane 110 portion of the contact lens device 112 contacts the eye 102. The inflatable bladder/reservoir 104 is designed to hold fluid, and can be filled with fluid to expand against and put pressure on the eye 102.

A hydraulic unit 124 is coupled via the hydraulic input/output 108 (hydraulic I/O) to the bladder/reservoir 104 to control the fluid inside the bladder/reservoir 104, and so control the pressure therein. In one embodiment, the hydraulic unit 124 is configured to measure pressure in the bladder/reservoir 104 or to work in conjunction with a pressure measurement system to measure pressure. The hydraulic unit 124 can also measure volume displacement inside the bladder 104. For example, the hydraulic unit 124 can include volume sensors or another volume detection/measurement apparatus that measures volume changes in the bladder over time. In one embodiment, the hydraulic unit 124 coupled to the bladder is configured to control the flow of fluid between the bladder/reservoir 104 and an external reservoir of the hydraulic unit 124 that holds fluid, so the hydraulic unit 124 can manage the filling of and removal of fluid from the bladder 104 as required by the system 100. The hydraulic unit 104 is illustrated in FIG. 1 as being separate from or external to the contact lens device 112, but in some embodiments, at least some components of the unit 104 (e.g., volume sensors or other components) are included within the device 112.

Although the dual-measurement system 100 can be implemented using a number of different designs, the soft contact lens design used in one embodiment system 100 allows pressure measurements to be easily taken in the clinical optometry environment. The flexibility and convenience of this hybrid, volume-adjustable, soft contact lens make it easy to be applied to the cornea surface, even by patients themselves. Thus, a tonography-style device or tonometer is implemented on a contact lens platform that takes measurements associated with the eye 102. In one embodiment, the contact lens device 112 uses pressure sensors 106 to take these measurements. In the FIG. 1a embodiment, the flexible surface 110 of the contact lens device 112 is embedded with pressure mapping sensors 106 for intraocular pressure detection. Since the flexible membrane 110 is brought into contact with the eye 102 when the contact lens is inserted into the eye 102, the sensors 106 are also placed into proximity with the eye 102. The sensors 106 in the flexible membrane 110 are configured to measure the pressure in the bladder/reservoir 104, allowing for noninvasive and convenient pressure and flow monitoring. In one embodiment, the pressure mapping sensors 106 function in conjunction with the external hydraulic unit 124 including external pressure and/or volume sensor(s). In some embodiments, the sensors 106 measure both pressure and volume changes. In other embodiments, the pressure and volume sensors are coupled to but are all external to the device 112.

The flexible contact membrane 110 is made of soft elastomer materials, such as silicone (Polydimethylsiloxane or PDMS), and the contact lens device 112 is backed by a relatively rigid outer shell 111 of polymeric materials, such as acrylic (Polymethyl-methacrylate or PMMA). A hydraulic chamber/reservoir 104 is enclosed in the shell, and is directly coupled to the ocular volume upon direct contact. The net change of the volumes in hydraulic chamber/bladder 104 and the anterior chamber 103 of the eye 102, which contains the aqueous humor or fluid to be measured, should be zero theoretically. Based on the volume correlation between the fluid in the bladder 104 and the aqueous humor in the eye 102, nanoliter volume displacement can be precisely monitored, e.g., through a computer-controlled interface.

The device 112 is also coupled via the electrical I/O 107 (e.g., wirelessly or wired) to a computer 122 or logic configured to process the measurements of the system 100, and a display 120 (e.g., a computer monitor or other type of information display mechanism). The computer 122 processes and stores pressure data and/or volume change data retrieved by the system 100, and the display 120 provides information to a user visually for user review or manipulation. The computer 122 can be used in calculating the outflow rate of fluid from the eye 102 based on the measured volume change of the eye 102 over time. The computer 122/display 120 represent the logic configured to compute the outflow resistance of the eye 102 as a function of the pressure in the bladder/reservoir 104 and the change of volume in the bladder 104 over time.

FIG. 1b illustrates the dual measurement system 100 for applying tonographic techniques to an eye, according to another embodiment. In this embodiment, the contact lens device 112 is designed in the same general manner as the device 112 shown in FIG. 1a, including an outer rigid wall/inelastic shell 111, a flexible inner membrane 110, and an adjustable hydraulic reservoir 104. The contact lens device 112 sits naturally on the cornea 152. In some embodiments, the overall footprint of the device 112 is greater than that of a regular contact lens, which covers the entire cornea surface and extends beyond the limbus area of the eye. The flexible inner membrane of the contact-lens device is in direct contact with the cornea surface, between which an ultrathin layer (e.g., a few microns) of tear film is left during the measurement.

In one embodiment, embedded nanocomposite pressure sensors (similar to those shown in FIG. 1a as 106) are incorporated into the device 112 as an additive feature for high-accuracy IOP measurement, though they are not required. The system 100 of FIG. 1b also includes a hydraulic unit 124 that takes the form of a computer-controlled nanofluidic pump including an external reservoir 156. In embodiments of FIGS. 1a and 1b, the external reservoir can be included in the hydraulic unit 124 or can be a separate entity coupled to the unit/pump 124. The unit/pump 124 is coupled to the bladder/reservoir 104 to control adding or removal of fluid from the reservoir 104. In one embodiment, the unit/pump 124 is coupled to the bladder/reservoir 104 via micro-tubing that manipulates the hydraulic volume of the lens device 112. Again, a computer 120/display 122 is illustrated in FIG. 1b that is in contact with the contact lens device 112, and functions in the same general manner as the computer 120/display 122 of FIG. 1a. The computer-controlled interface allows pressure information to be directly employed to control the hydraulic flow to the bladder/reservoir 104. The FIG. 1b embodiment further illustrates an external pressure sensor 160 that is in contact with the reservoir 104, and can also be in contact with the hydraulic unit/pump 124 and the computer 120.

### Method for Outflow Resistance Measurement

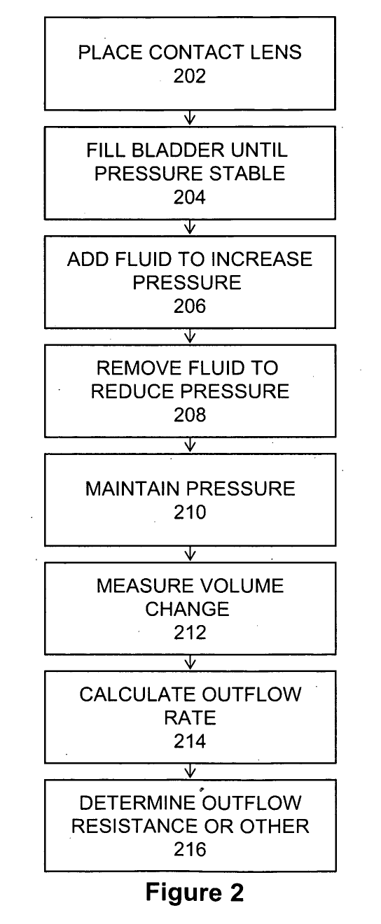

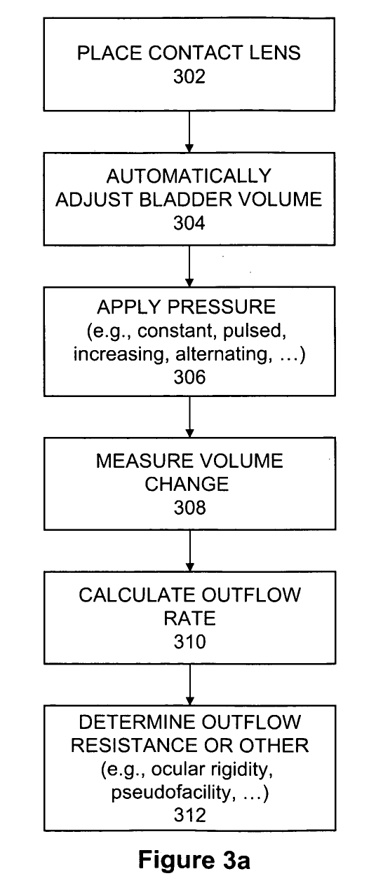

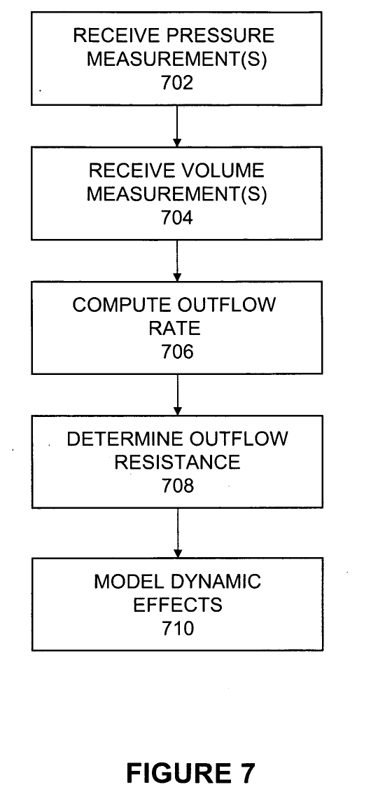

FIG. 2 is a flow diagram illustrating the method for measurement of the outflow resistance of an eye, according to some embodiments of the present invention. It should be understood that these steps are illustrative only. Different embodiments of the system 100 may perform the illustrated steps in different orders, omit certain steps, and/or perform additional steps not shown in FIG. 2 (the same is true for FIG. 3 and FIG. 7).

During operation of the contact lens device 112, the bladder/reservoir 104 is placed 202 in the eye 102. To take the IOP measurements, the contact lens 112 can be placed 202 in the eye 102 with topical anesthetic while the patient lies back & relaxes. One or both eyes can be tested at the same time. In the embodiment of FIG. 2, the system 100 is used to slowly fill 204 bladder 104 with fluid until the trans-membrane pressure signal is stable. Filling 204 the bladder 104 in this manner can initially expand the flexible inner wall against the eye a stable pressure is reached. This will be the baseline pressure (P_{baseline}). The pressure sensors 106 and/or 160 or other pressure measurement mechanism can measure the pressure applied to the eye (e.g., at set intervals or continuously) to determine when the system 100 has reached the stable pressure signal (e.g., the baseline pressure level).

The system 100 can then be used to increase the pressure applied to the eye 102 by adding 206 additional fluid to the bladder 104 to raise the IOP a fixed amount (e.g., P_{baseline}+20 mmHg) over the baseline pressure. The pressure sensors 106 and/or 160, or other pressure measurement mechanism can measure the pressure applied to the eye (e.g., at set intervals or continuously) to determine when the fixed amount of pressure is reached. The bladder can thus be brought to a pressure that exceeds the starting IOP of the eye 102, which further expands the flexible inner wall/membrane 110 against the eye 102 to place pressure on the eye 102.

In some embodiments, during the operational run of the system 100, the servo-controlled microfluidics maintain 208 the pressure level (e.g., P_{baseline}+20 mmHg) absolutely steady (±0.1 mmHg resolution/100 msec). In one embodiment, the hydraulic unit 124 increases or decreases fluid in the bladder to maintain/regulate the pressure on the eye 102 at this fixed amount for a period of time based on continuous pressure measurements by the pressure sensor(s) 106 and/or 160. In this manner, the system 100 can account for patient squeezing, valsalva (forceable exhalation against a closed airway, etc. and other outside forces that might otherwise interfere with the pressure readings. This increased pressure on the eye 102 is thus maintained 208 for a period of time, and the pressure on the eye 102 tends to cause fluid outflow from the eye 102 during this time.

After a pre-programmed time interval (e.g., 2 or 4 min or other time interval), the system 100 draws/removes 210 fluid from bladder 104 until the trans-membrane IOP returns to P_{baseline}. The system 100 thus decreases the pressure on the eye 102 to return the pressure to the baseline pressure level. The fluid outflow can be measured using the change in volume of the bladder 104 as a proxy for the change in volume of the eye 102 over time, assuming that the increased volume in the bladder 104 is directly related to a loss of volume of fluid in the eye 102. The volume needed to fill the bladder 104 at the end of the run to return the pressure reading to P_{baseline} (V2) minus the volume needed to fill the bladder at the start of the run (V1) represents the outflow volume during the run (current microfluidics technology allows this to be measured with 0.1 μL precision), so the change in volume—and thus, the outflow—has been measured 212 directly. In one embodiment, the passive pressure sensors 106 and/or sensor 160 coupled to the bladder 104 working with the hydraulic unit 124 (e.g., the volume sensors) can directly measure 212 the decreasing volume in the bladder 104 over time under the presence of a known, measured pressure. In one embodiment, the system 100 can take a plurality of measurements of the change in volume of the eye 102 over time under the increased pressure. During the procedure, time and perturbing pressure are tightly controlled. This operation can be performed on one eye or on both eyes simultaneously.

With the data collected, the time-dependent variation of ocular volume can be used to calculate 214 flow rate as Q=ΔV/Δt. The system 100 computes 214 the outflow rate of fluid from the eye 102 based on the measured volume change of the eye over time. The resistance can then be determined 216 as R=ΔP/Q. The system 100 thus determines 216 the outflow resistance of the eye as a function of the ratio of the applied pressure and the outflow rate. In some embodiments, the system 100 can also measure other ocular parameters, such as ocular rigidity, pseudofacility, or other ocular mechanical parameters related to flow or pressure.

In one embodiment, the data collected by the system 100 can be outputted to a display (e.g., computer display 122) for viewing and/or manipulation by the user. In addition, information regarding the computations 308 performed to determine the outflow rate or the determination 310 of the outflow resistance can be provided on the display 122. Similarly, the final results of the calculations/determinations 308/310 can be outputted on display 122 for the user to view/manipulate.

There can be a number of different variations on the method steps above. In some embodiments, step 208 (FIG. 2) of the method is optional, and the pressure does not have to be maintained 208 over time. For example, the pressure could be pulsed or fluctuating over time, and so not maintained at a constant level. As another example, the pressure could be gradually increased over time or could be alternating. The method can include various other pressure waveforms, as well. Further, in the embodiment described above, the method describes changing the pressure applied and measuring the resulting volume change. However, in other embodiments, the method includes changing the volume over time and measuring the pressure, as illustrated in FIG. 3b.

Referring now to FIGS. 3a and 3b, there are shown flowcharts illustrating the operation of the dual measurement system 100, according to other embodiments of the invention. Like, FIG. 2, the methods of FIGS. 3a and 3b include placing 302, 352 the contact lens in the patient's eye in a manner similar to that described for step 202 above. In some embodiments, the methods of FIGS. 3a and 3b include automatically adjusting the bladder volume. As one example, the hydraulic unit/pump 124 can automatically add fluid to the bladder 104 or remove fluid from the bladder 104 until the appropriate volume is reached.

Continuing with FIG. 3a, the method further includes applying 306 pressure to the eye 102. As one example, the hydraulic unit 124 can add fluid to the bladder 104 which is resting against the eye 102, causing the bladder 104 to apply 306 pressure to the eye 102. As explained above, the pressure applied can be constant, pulsed, increasing, alternating, etc. over time. As also explained above regarding FIG. 2, placing pressure on the eye 102 tends to cause fluid outflow from the eye 102. In step 308 of the method, the system 100 can directly measure 308 a volume change of the eye created by the applied pressure 306 to the eye over time. With the data collected, the system can calculate 310 the outflow rate of fluid from the eye 102 based on the measured volume change of the eye over time, and can determine 312 the outflow resistance of the eye or other ocular parameter (e.g., ocular rigidity, pseudofacility, etc.), as explained above regarding FIG. 2.

Returning to FIG. 3b, the method continues with the step of imposing 356 a volume change in the bladder. As one example, the hydraulic unit 124 can add fluid to the bladder 104 or remove fluid from the bladder 104 to impose this change. In some embodiments, the changing volume is constant, pulsed, increasing, alternating, or changed in some other pattern over time. The addition or removal of fluid from the bladder 104 causes the bladder 104, which is resting against the eye 102, to create changes in pressure applied to the eye 102. In one embodiment, the pressure sensors 106 and/or 160 or other pressure measurement mechanism can directly measure 358 the pressure change of the eye created by the imposed 356 volume change. With the data collected, the system can calculate 360 the outflow rate of fluid from the eye 102 based on the measured pressure change of the eye over time, and can determine 362 the outflow resistance of the eye or other ocular parameter (e.g., ocular rigidity, pseudofacility, etc.), as explained above regarding FIG. 2. Thus, using any of the methods of FIGS. 2, 3a, and 3b, the system can detect IOP and ocular volume change simultaneously, and can measure outflow resistance directly.

### Physical/Mathematical Model for the Intraocular Biomechanics and Microfluidic Dynamics

Background

To understand the mathematical model used by the dual measurement system 100, it is helpful to first review the current tonography procedures and their deficiencies. As explained above, current tonography procedures face an intrinsic technical hurdle. In order to measure flow resistance or facility, two measurable quantities are typically required: pressure drop (ΔP) and flow rate (Q) per volume change (ΔV/Δt). But in tonography, the only measurement made is through the reading of indentation level. Therefore, statistical correlations applied in tonography procedures relate the indentation levels to both volume change and pressure reading under a constant weight on the cornea surface. Formulas typically used in current tonography procedures include the following:

V₁=1/K_{T}*log(P_{T1}/P₀₁)

V₂=1/K_{T}*log(P_{T2}/P₀₂)→ΔV=K_{T}/K_{D}*(1/K_{T}*Log(P_{T1}/P_{T2})−V₂+V₁)

ΔV=1/K_{D}*log(P₀₁/P₀₂)

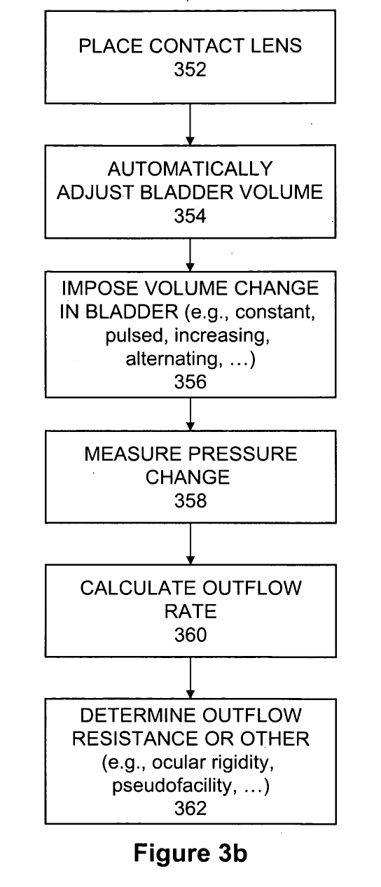

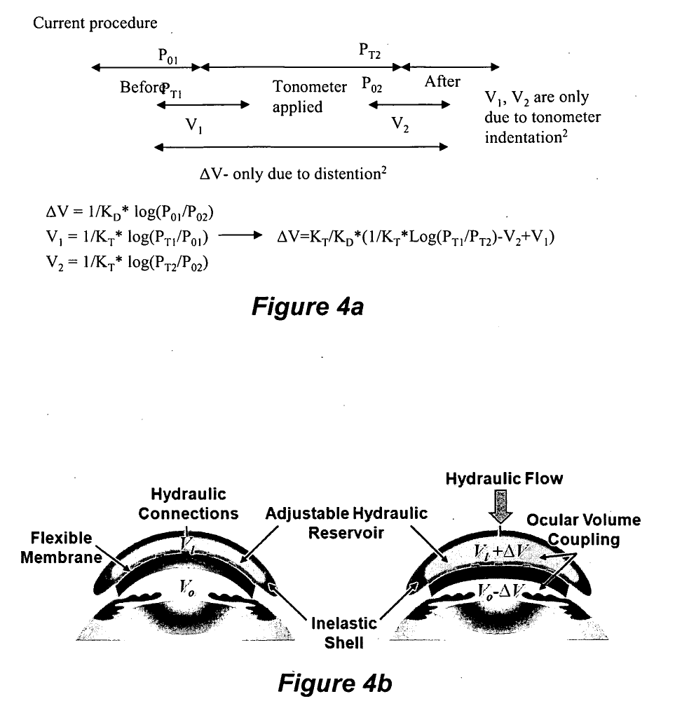

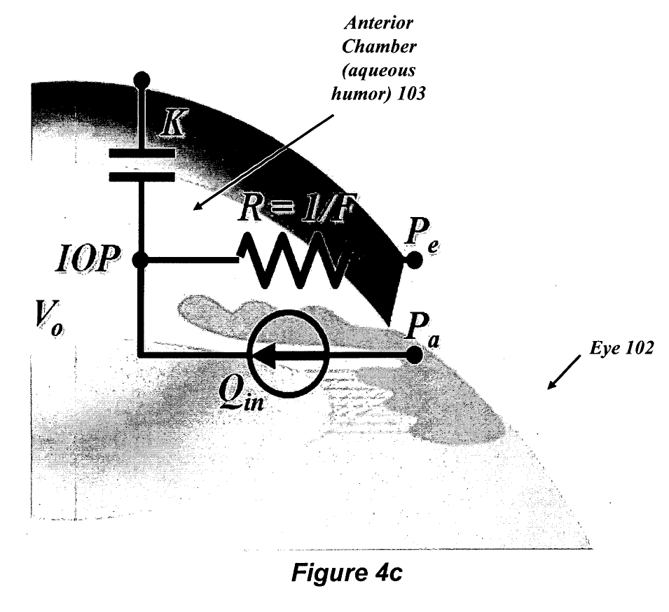

FIG. 4a depicts current tonography procedures in more detail. The total change in volume due to the application of the tonometer, ΔV, is calculated using the above formula. P₀₁ and P₀₂, are the pressures before and after application of the tonometer, respectively. P_{T1} and P_{T2} are the pressures during application of the tonometer at time point 1 and time point 2, and these values are obtained from standard tonometer calibration tables (e.g., open manometer calibration tables). V₁, V₂ are the volumes before and after tonometer application, respectively, and are also obtained from the calibration tables. The average values of K_{T} and K_{D} are used. See Grant W. M. Tonographic method for measuring the facility and rate of aqueous flow in human eyes. Archives of Opthalmology. 44:204-214 (1950), which is incorporated by reference. Finally, the facility of outflow C=(ΔV/T)/(P_{Taverage}−P₀₁)), where T is the time of application of the tonometer. P_{Taverage} is the average pressure over the tonometer application time. Low C values have been found in patients with glaucoma.

Problems with this approach include the fact that just one tonometer reading is used to determine both the numerator and the denominator (2 properties) in the formula for C. Moreover, the formula should read as: C=(ΔV/T)/(P−Pv), where Pv is the episcleral venal pressure and P is the intraocular pressure. The denominator is the pressure difference which is the driving force for the aqueous humor flow, and the numerator is the volumetric flow rate. C is therefore equivalent to the inverse of the resistance to this flow (compare with Ohm's law). Another issue is that P₀₁ and P₀₂ are obtained from closed manometer calibration, which is not reliable.

### Hybrid Dual-Parameter Measurement Principle

Rather than relying on measurement of the cornea/sclera deformation under a mechanical load like conventional ocular biomechanical assessments, the dual-parameter (ΔIOP-ΔV) measurement system 100 couples, manipulates and continuously measures both ocular volume and IOP change. To accurately evaluate flow resistance (R) or facility (F) in any linear fluidic system, two measurable quantities are typically required, the pressure difference (ΔP) and the according outflow rate (Q) or volume change rate (ΔV/Δt), as indicated in the definition of flow resistance or facility in Equation 1:

$$R = \frac{1}{F} = \frac{Δ F}{q} = \frac{Δ F}{Δ V / Δ t} \qquad ( 1 )$$

FIG. 4b is an illustration of the dynamic, dual-parameter measurement (a) prior to and (b) after the ocular volume change, according to an embodiment. As shown in part (a), this configuration allows direct coupling of the adjustable fluidic reservoir in the contact lens to the anterior chamber. The two-way nanoliter-precision hydraulic pump 124 perfuses liquid into or out from the contact-lens reservoir 104, which displaces the complementary volumes of the reservoir (Vₗ) and anterior chamber (Vₒ) simultaneously (part (b) of FIG. 4b) following the relationship described in the Equation 2:

Vₗ+Vₒ=constant or ΔVₗ+ΔVₒ=0 (2)

By adjusting the ocular volume while continuously monitoring the IOP, the pressure/volume relationship (ΔIOP-ΔV) of the eye is established dynamically, enabling determination of the aqueous outflow resistance/facility.

Dynamic Dual-Parameter Measurement Modeling

To understand the intraocular biomechanics coupled with fluidic dynamics of aqueous humor (the circulation flow inside the anterior chamber), a mathematical/biomechanical model has been developed and can be used in conjunction with the dual-measurement system 100. The dynamic, dual-parameter concept is similar to the impedance analysis in circuits, where a tiny current excitation is produced to generate a measurable voltage shift. A lumped-element model, analogous to an electronic circuit model, has been developed to understand the intraocular biomechanics coupled with fluid dynamics of aqueous humor, the circulation flow inside the anterior chamber. FIG. 4c shows the lumped-element model of the aqueous humor hydrodynamics for the proposed dynamic measurement techniques, according to an embodiment. As the Figure shows, it is the linear component of the aqueous outflow resistance (R) that is the primary measurand in the model, while the ocular rigidity of the eye (K), a compliance measure of the corneosclera envelope, is also included. The governing equations can be derived from Equation 1 and the biomechanical model for corneoscleral envelope.

$$\begin{matrix}R = \frac{d ( IOP )}{d ( \frac{d V}{d t} )} & ( 3 ) \\ K = \frac{( d IOP / d t ) / ( d V / d t )}{IOP} & ( 4 )\end{matrix}$$

Using similar approaches to the circuit analysis (Kirchhoff's current and voltage laws), the equations of conservation of mass and energy are employed to establish relationships between the ocular flows and pressures in the hybrid fluid mechanical model. Furthermore, to clinically exam the unknown ocular parameters, in particular, the outflow resistance, various excitation schemes can be explored in the dual-parameter measurement system 100. The simplest operation schemes are the constant-flow mode and constant-pressure mode. Unlike those used in the current tonography method, the constant-pressure mode employs an invariant pressure greater than the IOP, which is applied onto cornea. Meanwhile, the coupled volume displacement of the eye is closely manipulated via the hydraulic interface of the lens. Finally, the evaluation outcomes can be used to compare with the tonography results.

Considerations for Measurement Accuracy

To guarantee accurate measurement of aqueous outflow resistance, several possible clinical issues should be considered. First, to ensure a direct and close coupling between the deformable reservoir 104 and the anterior chamber 103, the contact-lens device 112 is held in place by the patient's eyelids in a manner similar to conventional techniques of clinical retinal electrophysiology while the pressure/volume change is applied. Meanwhile, the thin tear film/membrane will induce considerable capillary adhesion (e.g., up to 200 mmHg) between the lens and the ocular surface, according to the Laplace's equation. Moreover, the flexible membrane of the lens is relatively unresistant to the pressure change, and highly adaptive to the cornea surface with slightly varied dimensions and curvatures. The altered ocular volume is relatively small in comparison with the entire volume of the anterior chamber, under which linear biomechanical analysis can be performed. Furthermore, due to existing stress in the cornea, the pressure assessed through the hydraulic reservoir 104 may not reflect the true IOP reading. Fortunately, according to the dynamic dual-parameter measurement (as illustrated in Equations 3 and 4), the IOP change, instead of absolute IOP value, is the primary concern. Under a small volume change of the anterior chamber (e.g., <2%), the measured pressure change is expected to reflect the IOP variation in the anterior chamber, which has been demonstrated the in vitro experimental investigation described below.

### Computer Product for Outflow Resistance Measurement

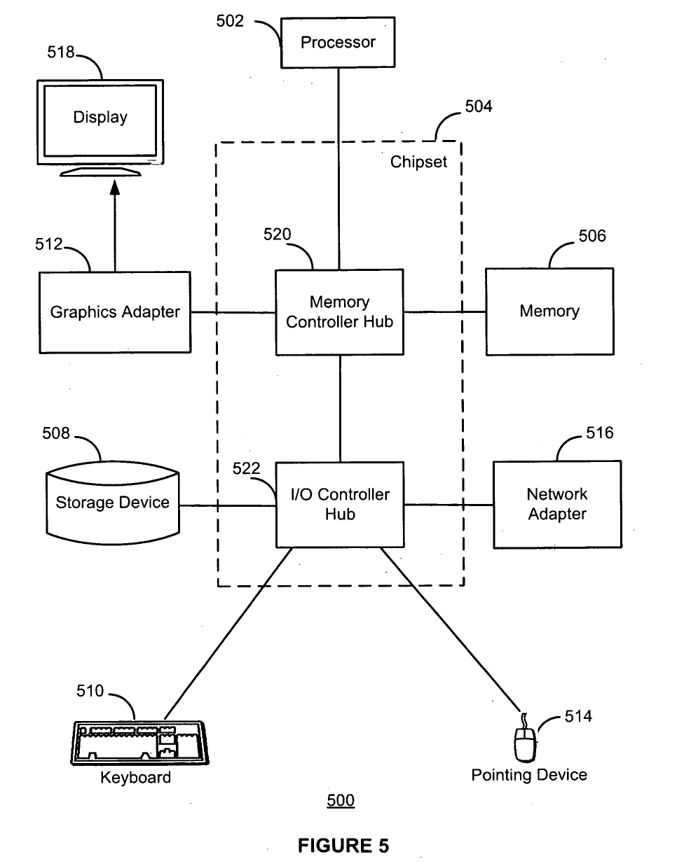

Embodiments of the invention can include a computer product that uses this biomechanical model described above. FIG. 5 is a high-level block diagram illustrating an example of a standard computer 500 for use with the computer product. Illustrated are at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display device 518 is coupled to the graphics adapter 512. A storage device 508, keyboard 510, pointing device 514, and network adapter 516 are coupled to the I/O controller hub 522. Other embodiments of the computer 500 have different architectures. For example, the memory 506 is directly coupled to the processor 502 in some embodiments.

The storage device 508 is a computer-readable storage medium such as a hard drive, compact disk read-only memory (CD-ROM), DVD, or a solid-state memory device. The memory 506 holds instructions and data used by the processor 502. The pointing device 514 is a mouse, track ball, or other type of pointing device, and is used in combination with the keyboard 510 to input data into the computer system 500. The graphics adapter 512 displays images and other information on the display device 518. The network adapter 516 couples the computer system 500 to a network. Some embodiments of the computer 500 have different and/or other components than those shown in FIG. 5.

The computer product may be performed or implemented with one or more hardware or software modules, alone or in combination with other devices. Thus, the computer 500 is adapted to execute the biomechanical modeling module 600 for providing functionality described. In one embodiment, a software module is implemented with a computer program product comprising a computer-readable medium containing computer program code, which can be executed by processor 502 for performing any or all of the steps, operations, or processes described. Embodiments of the invention may also relate to an apparatus for performing the operations herein. This apparatus may be specially constructed for the required purposes, and/or it may comprise a general-purpose computing device selectively activated or reconfigured by a computer program stored in the computer 500. Such a computer program may be stored in a tangible computer readable storage medium (e.g., storage 508) or any type of media suitable for storing electronic instructions, and coupled to a computer system bus. Furthermore, any computing systems referred to in the specification may include a single processor or may be architectures employing multiple processor designs for increased computing capability. In addition, the computer 500 can take the form of another electronic device, such as a personal digital assistant (PDA), a mobile telephone, a pager, or other devices. The computers can execute an operating system (e.g., LINUX®, one of the versions of MICROSOFT WINDOWS®, and PALM OS®), which controls the operation of the computer system, and execute one or more application programs.

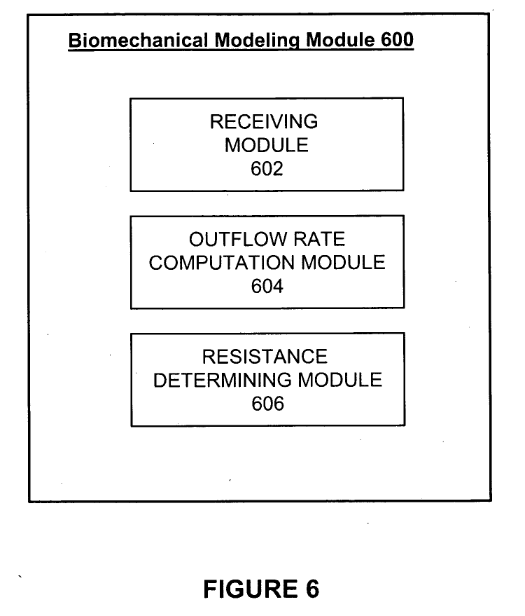

In one embodiment, the computer product is executed as a biomechanical modeling module 600, shown in FIG. 6. FIG. 6 is a high-level block diagram illustrating the functional modules associated with the biomechanical modeling module 600, according to one embodiment of the invention. In the embodiment illustrated in FIG. 6, the biomechanical modeling module 600 includes a receiving module 602, an outflow rate computing module 604, and a resistance determining module 606. Some embodiments have different and/or additional modules than those shown in FIG. 6 and the other figures. Likewise, the functionalities can be distributed among the modules in a manner different than described herein or can be incorporated into other modules.

The receiving module 602 receives the pressure measurement representing the applied pressure to the eye 102. The receiving module 602 also receives the set of volume measurements representing the directly measured volume change of the eye 102 at a plurality of times. In one embodiment, the pressure measurement and volume measurements are obtained via a tonographic-style device, such as a contact lens device 112 similar to that illustrated in FIGS. 1a and b (though other mechanisms could be used to obtain these measurements). The measurements can be obtained automatically from the device 112 (e.g., wirelessly) or via manual input by a user into a computer, such as computer 122 in FIGS. 1a and b. These measurements can be obtained through placement of the device 112 in the eye and filling of the bladder 104, according to the method steps described in FIGS. 2 and 3. In one embodiment, pressure measurements are taken via one or more pressure sensors 106 and/or 160 associated with the inflatable bladder 104 of the contact lens device 112 as described above, and transmitted to module 602 for analysis. Similarly, the volume measurements can be taken by the hydraulic unit 124 (e.g., volume sensors) working in conjunction with the pressure sensors 106 and/or 160 as described above, and transmitted to module 602 for analysis.

The outflow rate computation module 604 computes an outflow rate of fluid from the eye 102 based on the measured volume change of the eye 102 over time. Where a device such as contact lens device 112 is used to obtain the pressure and volume measurements described above, the pressure sensors 106 and/or 160 coupled to the bladder 104 in conjunction with the hydraulic unit 124 can directly measure the decreasing volume over time under the presence of a known, measured pressure. Module 604 can compute the outflow rate using this change in volume of the bladder 104 as a proxy for the change in volume of the eye 102 over time, as explained in more detail above.

The resistance determining module 606 determines the outflow resistance of the eye 102 as a function of the ratio of the applied pressure and the outflow rate. The resistance can be determined as R=ΔP/Q. The module 606 also uses the biomechanical model of the eye 102 described in detail above to model dynamic effects.

Referring now to FIG. 7, there is shown a flowchart illustrating the operation of the biomechanical modeling module 600, according to some embodiments of the invention. The module 600 receives 702 a pressure measurement representing the pressure applied to the eye 102. The module 600 also receives 704 a set of volume measurements representing the directly measured volume change of the eye at a plurality of times. These measurements can be taken using a device, such as contact lens device 112, and can be provided to module 600 automatically or via user input. The module 600 further computes 706 an outflow rate of fluid from the eye 102 based on the measured volume change (e.g., measured via contact lens device 112) of the eye over time. In addition, the module 600 determines 708 the outflow resistance of the eye as a function of the ratio of the applied pressure and the outflow rate, and uses the biomechanical model of the eye 102 to model 710 dynamic effects, as described in more detail above. The data collected by the system 100 and processed by the biomechanical modeling module 600 can be outputted to a display (e.g., computer display 122) for viewing and/or manipulation by the user. In addition, information regarding the computations 706 performed to determine the outflow rate or the determination 708 of the outflow resistance can be provided on the display 122. Similarly, the final results of the calculations/determinations 706/708 can be outputted on display 122 for the user to view/manipulate.

### Fabrication of the Contact Lens Device

Below is an example of specific embodiments for fabricating contact lens device 112. The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

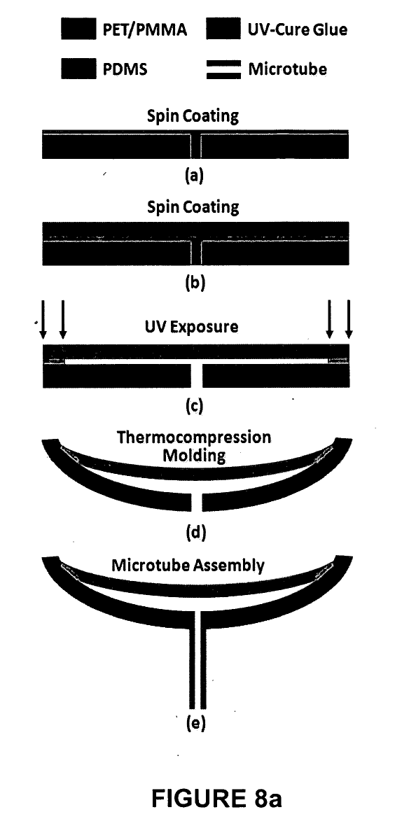

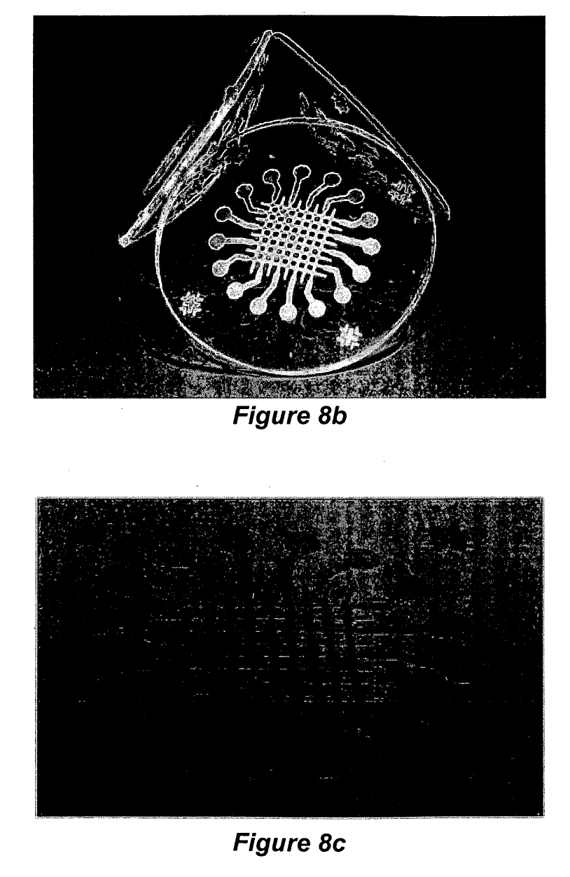

The contact lens device 112 can be fabricated in a number of different manners, and by using various different materials. The device 112 integrates microfluidic control and pressure sensing capacity into a hybrid contact-lens platform to evaluate aqueous outflow resistance accurately. FIG. 8a is an illustration of the microfabrication of the contact lens device, according to an embodiment. The Figure illustrates the microfabrication process for a smart contact-lens device 112. A silicone elastomer (e.g., PDMS) is used as the construct for the flexible membrane. PDMS has high optical transparency, high mechanical flexibility, excellent biocompatibility and easy processability. In particular, its Young's modulus is more than 10 times smaller than that of the corneoscleral envelope, providing high adaptability to cornea surface and low resistance to pressure/volume change. An array of miniature pressure sensors can be into this material, as described below.

On the outer shell, a much stiffer biocompatible polymer (e.g., PET or PMMA), is used, which ensures one-way volume expansion under positive pressure. A spinnable ultraviolet-curable adhesive (e.g., LOCTITE FLASHCURE®) is used to define the hydraulic volume and seal the PDMS membrane to the plastic shell. Thickness of the flexible membrane and adhesive layer can be controlled by spinning coating, which results in the target thickness of 80 μm and 20 μm, respectively. The rigid shell of 100 μm in thickness can be purchased from the manufacturer (e.g., DUPONT®) directly. Thus, the overall thickness of 200 μm for the contact lens device 112 is similar to that of a vision-correction contact lens, with an entire footprint of 2 cm in diameter to completely cover the cornea surface for accurate volume coupling from the contact lens to anterior chamber. In the subsequent thermocompression molding, the device 112 is shaped into a spherical dome to match with the cornea curvature under an elevated temperature (the glass transition temperature) and a mechanical pressure (part (d) of FIG. 8a). Finally, a microtube is glued to the through-hole of the inelastic shell (part (e) of FIG. 8a).

In some embodiments, very flexible, nanocomposite sensors (e.g., sensors 106) are embedded in the device 112 (e.g., as an additive monitoring feature to achieve higher accuracy for the IOP measurement). The sensors are fabricated using a photopatternable, conductive, nanocomposite polymer comprising conductive filler (e.g., silver nanoparticles) and an additional photosensitive component well dispersed into an elastomer matrix (e.g., PDMS). The PDMS-Ag nanocomposite material provides very high electrical and thermal conductivity, along with enhanced mechanical strength. The built-in photopatternability makes manufacturing process easy and very reproducible. FIGS. 8b and 8c are photographs of a prototype of an array of miniature pressure sensors fabricated onto a flexible contact-lens platform, according to an embodiment. In one embodiment, the array of miniature pressure sensors is fabricated onto a flexible contact lens platform using the nanocomposite.

Fabrication of the pressure sensing elements on the flexible membrane begins with mixing of a commercially available PDMS base with a curing agent in a 10:1 (w/w) ratio. The silicone pre-polymer is spin-coated onto a 4 inch silicon substrate at 1,000 rpm. The PDMS membrane of about 60 μm thick is thermally cured at 80° C. for one hour. The photosensitive conductive nanocomposite material is prepared from the PDMS prepolymer mixture with Benzophenone (3 wt %), the photosensitizer, and silver nanopowder (21 vol %, 150 nm in diameter), the conductive filler. It is spin-coated onto the cured pure PDMS film at 4,000 rpm to achieve a 20 μm-thick layer. The spin-coated substrate is ultraviolet exposed under a chrome photomask using proximity mode (of 50 μm separation). Unlike the regular photosensitive polymers, the conductive PDMS-Ag nanocomposite requires a heavy exposure dosage (˜7000 mJ/cm²), possibly resulting from strong ultraviolet absorption and scattering by silver nanoparticles present in the film. Subsequently, a post-exposure bake is carried out at 120° C. for 50 sec, which facilitates the further crosslink in the unexposed region. The exposed PDMS-Ag composite is then removed in toluene for 3-5 sec during the development. Finally, the wafer is rinsed with 2-propanol and blow-dried under nitrogen flow.

After fabrication of the conductive polymeric circuits, an ultrathin PDMS layer is spin-coated on top of the surface at 5,000 rpm. This PDMS layer of 12 μm thick, only half cured for the following folding bond process, serves as a pressure sensitive layer in the capacitive sensing design. Subsequently, the elastomer sensing circuit membrane is folded over and fully thermally cured to secure final packaging. The sensing circuits on each side are orthogonally crossed over and form a matrix of capacitive sensing elements in the film. At the end, a thermal compression process on a curved surface is used to form the final contact lens shape, as shown in FIGS. 8b and 8c.

### In Vitro Bench Test to Evaluate Aqueous Flow Resistance

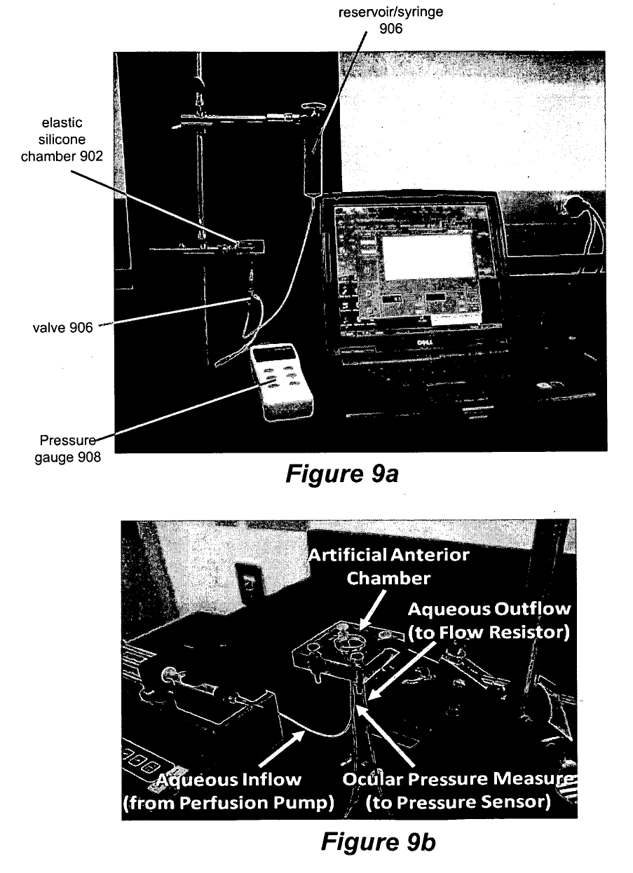

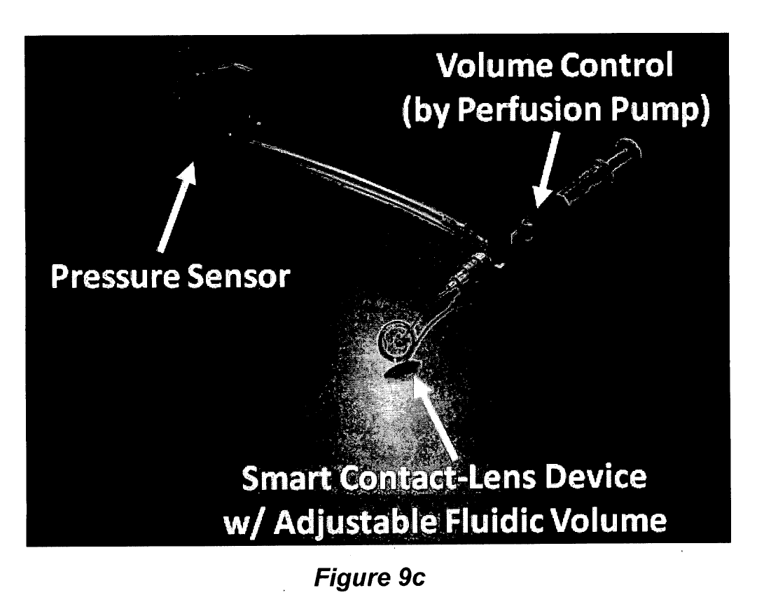

FIG. 9a shows an in vitro biomechanical experiment setup that was built to evaluate the aqueous flow resistance. This example is provided to illustrate, through an in vitro apparatus, how the system 100 might function in vivo. This example (and the other examples below) is not intended to limit the scope of the invention in any way.

An elastic silicone chamber 902 with a deflectable membrane was constructed to simulate anterior chamber 103 and cornea surface. A manometer reservoir/syringe pump 904 providing a flow stream to the simulated anterior chamber 103 is connected to the inlet of the eye model through a three-way valve 906, the other end of which directs to a computer-controlled pressure gauge 908. The outlet of the chamber passes to a flow restrictor, which provides a linear resistance to the flow. Using the same setup, the plastic anterior model can be replaced with a cadaver eye. Based on the findings from the biomechanical analysis, the in vitro example can be used to optimize measurement design on displaced ocular volume and/or intraocular pressure.

FIG. 9b shows a photo of another in vitro biomechanical experiment setup that was built to simulate the anterior chamber with aqueous humor circulation. The FIG. 9b setup is similar to the FIG. 9a setup, including artificial anterior chamber (like chamber 902), an aqueous outflow tube leading to a flow resistor, an aqueous inflow tube coming from a perfusion pump, and an ocular pressure measure to a pressure sensor. The artificial anterior chamber model of FIG. 9b is constructed from an acrylate polymer, which consists of a fluidic chamber in the plastic substrate, a clamping sleeve, and an artificial cornea manufactured by silicone rubbers with a similar Young's modulus to the human cornea. The designed cavity is 12 mm in diameter and 3 mm in depth and, together with the mounted artificial cornea, forms the artificial anterior chamber, of which the volume is about the same size as that in vivo. Four plastic screws secure the seal from the cornea under the clamping sleeve to the artificial anterior chamber. Furthermore, the fluidic chamber contained three through-channels from the backside. Among the three, two channels provide an inflow path from a digital perfusion pump and outflow drainage to a reservoir, respectively, and the other allows real-time tracking on hydraulic pressure inside the chamber by a digital pressure sensor.

On the outflow path, a microfluidic channel is connected to mimic the flow resistance to the aqueous outflow. The flow resistance (R) can be designed according to the geometric dimensions and fluidic viscosity, as shown in the Poiseuville's equation:

$$R = \frac{8 μ l}{π {r}^{4}} \qquad ( 5 )$$

where l and r indicates the length and radius of the microfluidic channel, respectively, while μ is the viscosity of the fluid. Under physiological conditions, the perfusion pump is operated at a constant flow rate of 45 nL/s (2.7 μL/min). In order to generate an artificial IOP of 2000 Pa (15 mmHg), the aqueous outflow resistance is set at 4.4×10¹³N-s/m⁵, which is used as the key design parameter for the flow resistor. Furthermore, the measured pressure changes in the contact-lens reservoir are directly compared with the true value measured by the pressure sensor connected to the inside chamber. Although little difference between the external and internal pressure variations has been experimentally observed under a small volume change of the anterior chamber (<2%), this configuration allows the further calibration of differential pressure measurements in the contact-lens device for a higher accuracy.

### Prototype of the Dual Measurement System

FIG. 9c is photograph of a prototype of the dynamic dual measurement system using the volume-adjustable contact-lens device 112, according to an embodiment. Through the micro tubing connection, the contact-lens device 112 with the embedded adjustable hydraulic bladder is connected to a three-way stopcock, one limb of which passes to a high-precision pressure sensor, and the other to a two-way high-precision perfusion pump, as described above. Then, both of the pressure sensor and nanofluidic pump are interfaced with a laptop computer. The two-way nanofluidic pump with cyclic infusion/withdrawal capacity (e.g., KD SCIENTIFIC 210) offers nanoliter-precision pulse-free flow to/from the hydraulic volume, which is microprocessor-controlled through the built-in TTL and RS232 interfaces. Similarly, the high-precision digital pressure sensor (e.g., OMEGA HHP 90) allows direct assessment of the hydraulic pressure in the deformable contact lens through the built-in RS232 interfaces. Furthermore, since the computer has digital access to both hydraulic flow control and pressure measurement, a software control interface uses the pressure information, which can be employed to manipulate the hydraulic flow through feedback. This interface would enable true dynamic dual-parameter measurement schemes, under which pressure-controlled excitation modes can be realized in the aqueous outflow resistance/facility assessment, e.g., the constant-pressure mode.

### Ex Vivo Investigation of the Dual Measurement System

The integrated hybrid measurement system and computer-controlled interface can be validated both ex vivo and in vivo. Porcine eyes can be used since they are comparable in size to human eyes. The scale of the prototype can thus be designed to be a similar size to a device designed for clinical use. By slightly modifying the in vitro validation model, an enucleated porcine eye can be immobilized with the cornea facing upwards. Subsequently, the anterior chamber is cannulated and infused with a simulated aqueous flow at a physiological rate driven by the perfusion pump. Meanwhile, the true IOP pressure in the cannulated eye can be measured directly through a three-way stopcock using the similar setup presented in FIG. 9b. The outflow resistance for each individual eye can be determined by this procedure first. Afterwards, the device is mounted on the eye surface, which is treated with artificial tears right before. The dynamic, dual-parameter assessment can be performed using various computer-directed operation schemes, including the constant-pressure and constant-flow modes, and the results can be directly compared with the measurements made through the invasive cannula system described above to determine the optimal protocol and conditions. In addition, eye movement and eyelid squeezing can be simulated by touching and pressing on the eye globe during the measurement, for analyzing influences from the environmental disturbances, and for designing strategies to minimize the extrinsic factors.

### In Vivo Validation of the Dual Measurement System

In vivo experiments can also be performed on anesthetized pigs. In a manner similar to that described above, the baseline outflow resistance in anesthetized pigs is measured through an invasive cannula system, where an artificial inflow is imposed, while the IOP is assessed by connecting to a three-way stopcock. The device described previously is sized appropriately to fit under the eyelids of a pig. During the hybrid measurement operation, the natural aqueous inflow occurring in the anesthetized animals can be assessed dynamically, instead of the simulated flow from the perfusion pump. The optimal testing protocol and parameters can be refined using the in vivo model. Moreover, in vivo IOP is a dynamic physiological parameter, influenced by eye movement as well as the ocular pulse. Various pulsed stimulations (either flow or pressure) can be used to determine whether dampening or simulating the existing ocular influences is necessary during the in vivo measurement.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. Accordingly, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon.

1. A method for measuring an outflow resistance of an eye, the method comprising:
applying a pressure to an eye;
measuring the applied pressure;
directly measuring a volume change of the eye created by the applied pressure to the eye;
computing an outflow rate of fluid from the eye based on the measured volume change of the eye over time; and
determining the outflow resistance of the eye as a function of a ratio of the applied pressure and the outflow rate.

2. The method of claim 1, further comprising
initially applying pressure to the eye until a stable pressure signal is received; and
measuring the pressure applied to the eye to reach the stable pressure signal, the pressure applied being a baseline pressure level.

3. The method of claim 2, wherein applying pressure to the eye further comprises:
increasing the pressure on the eye to raise intraocular pressure a fixed amount; and
maintaining the pressure on the eye at this fixed amount for a period of time.

4. The method of claim 3, further comprising decreasing the pressure on the eye to return the pressure to the baseline pressure level, wherein the volume change is measured at the baseline pressure level.

5. The method of claim 3, further comprising taking a plurality of measurements of the change in volume of the eye over time under the increased pressure.

6. The method of claim 1, further comprising:
placing a pressure sensor in proximity to the eye;
continuously measuring the applied pressure detected by the pressure sensor; and
regulating the applied pressure using the pressure sensor to maintain the applied pressure at a stable level.

7. The method of claim 1, wherein the outflow resistance and an ocular rigidity of the eye are determined using mathematical modeling and experimental measurements from a pressure sensor placed in proximity to the eye.

8. The method of claim 1, further comprising:
placing a contact-lens device in the eye, the contact-lens device comprising a rigid outer wall, a flexible inner wall, and an inflatable bladder disposed therebetween, wherein the flexible inner wall contacts the eye and is coupled to a pressure sensor for measuring pressure applied to the eye; and
filling the bladder with fluid until a stable pressure signal is received representing a baseline pressure level.

9. The method of claim 8, wherein applying pressure to the eye further comprises:
filling the inflatable bladder with additional fluid to increase the pressure on the eye to raise intraocular pressure a fixed amount; and
increasing or decreasing fluid in the bladder to maintain the pressure on the eye at this fixed amount for a period of time based on continuous pressure measurements by the pressure sensor.

10. The method of claim 9, further comprising removing fluid from the inflatable bladder to decrease the pressure on the eye to return the pressure to the baseline pressure level, wherein the volume change in the bladder is measured at the baseline pressure level, the volume change in the bladder representing the volume change in the eye.

11. The method of claim 1, further comprising applying directly measured intraocular pressure change and directly measured volume change of the eye to determine a plurality of different ocular mechanical parameters related to flow or pressure of the eye.

12. A system for measuring an outflow resistance of an eye, the system comprising:
a contact-lens device comprising a rigid outer wall, a flexible inner wall, and an inflatable bladder disposed therebetween, the contact-lens device having a concave shape to allow placement over an eye wherein the flexible inner wall contacts the eye;
a pressure measurement system coupled to the bladder and configured to measure a pressure of fluid within the bladder and applied to the eye;
a hydraulic unit coupled to the bladder and configured to control a flow of fluid between the bladder and an external reservoir, and further configured to measure a change of volume in the bladder created by the pressure applied to the eye; and
logic configured to compute the outflow resistance of the eye as a function of the pressure in the bladder and the change of volume in the bladder over time.

13. The system of claim 12, wherein the pressure measurement system comprises a pressure sensor embedded in the flexible inner wall of the contact-lens device for directly measuring the pressure of fluid within the bladder.

14. The system of claim 12, wherein the pressure measurement system comprises a pressure sensor external to and coupled with the contact-lens device.

15. The system of claim 12, wherein the hydraulic unit is configured to control filling of the bladder with fluid to increase pressure on the eye and is configured to control removal of fluid from the bladder to decrease pressure on the eye.

16. The system of claim 12, wherein the hydraulic unit comprises a volume sensor for directly measuring change in the volume of fluid in the bladder as a proxy for fluid outflow from the eye, the hydraulic unit being coupled to the bladder via micro-tubing through which fluid flows to and from the bladder.

17. The system of claim 12, wherein the logic further comprises logic for using a biomechanical model of the eye to model dynamic effects, the model being used in conjunction with experimental data to determine the outflow resistance and an ocular rigidity of the eye.

18. The system of claim 12, further comprising a computer interface for monitoring nanoliter volume displacement in the eye, represented by volume change in the bladder over time.

19. A computer program product for measuring an outflow resistance of an eye, the computer program product comprising a computer-readable storage medium containing computer program code that comprises:
receiving a pressure measurement representing an applied pressure to an eye;
receiving a set of volume measurements representing a directly measured volume change of the eye created by the applied pressure to the eye;
computing an outflow rate of fluid from the eye based on the measured volume change of the eye over time; and
determining the outflow resistance of the eye as a function of a ratio of the applied pressure and the outflow rate, and using a biomechanical model of the eye to model dynamic effects.

20. The computer program product of claim 19, wherein the model uses the set of volume measurements and the pressure measurement to calculate the outflow resistance or facility of outflow and an ocular rigidity of the eye.

21. The computer program product of claim 19, wherein receiving the pressure measurement further comprises receiving the pressure measurement from a device with an inflatable bladder placed in the eye having a flexible membrane contacting the eye, the device being coupled to a pressure sensor for measuring the applied pressure.

22. The computer program product of claim 19, wherein the volume measurements received are based on a change in volume of fluid in the inflatable bladder as a proxy for a change in volume of fluid in the eye over time.
